(19) United States
(12) Patent Application Publication
(10) Pub. No.: US 2022/0183922 A1
(43) Pub. Date: Jun. 16, 2022

(54) MASSAGE CHAIR HAVING AN IMPROVED ARMREST AND SHOULDER SLIDING MECHANISM, WIRELESS CHARGER, HAMMERING DEVICES, AND OXYGEN GENERATION

(71) Applicant: Luraco, Inc., Arlington, TX (US)

(72) Inventors: Kevin LE, Arlington, TX (US); Thanh LE, Arlington, TX (US); Varad Nitin GOKHALE, Santa Clara, CA (US)

(73) Assignee: Luraco, Inc., Arlington, TX (US)

(21) Appl. No.: 17/509,051

(22) Filed: Oct. 24, 2021

Related U.S. Application Data
(63) Continuation-in-part of application No. 16/439,218, filed on Jun. 12, 2019, now Pat. No. 11,179,290.
Application No. 16/439,218 is a continuation of application No. 16/283,186, filed on Feb. 22, 2019, now abandoned.
Application No. 16/283,186 is a continuation-in-part of application No. 15/593,320, filed on May 11, 2017, now Pat. No. 10,724,549.
Application No. 15/593,320 is a continuation-in-part of application No. 15/593,320, filed on May 11, 2017, now Pat. No. 10,724,549.

(51) Int. Cl. A61H 9/00; H02J 50/12; A47C 1/00; A61H 23/00; A61H 39/06; A61H 31/00; A61H 39/00

(52) U.S. Cl. CPC A61H 9/0071; A61H 2201/1638; A47C 1/00; A61H 9/0078; A61H 23/006; A61H 39/06; A61H 31/00; A61H 9/00; A61H 39/00; A61H 2201/5048; A61H 2201/0149; A61H 2201/5046; A61H 2201/5007; A61H 2201/1633; A61H 2203/0431; A61H 2201/1207; A61H 2201/5082; A61H 2201/169; A61H 2201/1623; A61H 2201/5097; A61H 2201/1642; A61H 2201/0169; A61H 2201/0207; H02J 50/12

(57) ABSTRACT

The present invention is directed to a massage chair having a massage chair frame, a massage system, and a noise-reducing, enclosure device. The frame includes a first end, a second end, a seat body area portion, and a back body area portion. The massage system includes at least one air massage element, an air pump, and at least one air valve device for regulating air flow into and out of the air massage element. The enclosure device includes an enclosure housing and noise-reducing material positioned inside the housing. The housing encloses the air pump and air valve device(s) during operation such that noise generated from or made by the air pump and air valve device(s) during operation is reduced, contained or eliminated. The present invention may include additional features: armrest sliding, shoulder airbag sliding, media player holder, virtual reality player, wireless charger, hammering devices, heating acupuncture devices, and oxygen generator.

### CROSS REFERENCE TO PRIOR APPLICATIONS

This application is a continuation in part of U.S. patent application Ser. No. 16/439,218, filed Jun. 12, 2019 and now allowed on Oct. 12, 2021, which is a continuation of U.S. patent application Ser. No. 16/283,186, filed Feb. 22, 2019 and now abandoned, which is a continuation in part of U.S. patent application Ser. No. 15/593,320, filed May 11, 2017 and now issued on Jul. 28, 2020 as U.S. Pat. No. 10,724,549, which are all incorporated herein by reference in their entireties.

### BACKGROUND OF THE DISCLOSURE

### Field of the Invention

The disclosure generally relates to massage chairs, massage devices and apparatuses for massage chairs, armrest sliding or swing, shoulder airbag sliding, media player holder, virtual reality player, and noise-reducing devices and systems. More specifically, the disclosure is directed to a massage chair having a noise-reducing, enclosure device. Additionally, the disclosure is directed to a massage chair having hammering devices. Moreover, the disclosure is directed to a massage chair having heated acupuncture devices. Additionally, the disclosure is directed to an oxygen generator for generating oxygen for user. Moreover, the disclosure is directed to a massage chair having a remote control pouch or holder and a wireless device pouch or holder having wireless charging functionality. Additionally, the disclosure is directed to voice recognition control of the massage chair.

### Description of the Related Art

Massage chairs, massage devices and apparatuses for massage chairs having sliding or swing armrest, shoulder airbag sliding, media player holder, virtual reality player, and noise-reducing devices and systems are known in the art. Many massage chairs are known for having two armrests fixed to the chair. This limitation gives the user, especially elderly people, difficulty in getting into the chair. Having two armrests sliding back or swing open will make it more easier and/or safer to gain access to the massage chair. The improved armrest can be slid or swung by the user's hand or by an electric actuator.

In many current massage systems and devices related to massage chairs, the massage system or device usually has an air compressor to pump air into the air cells or bags throughout the massage chair to provide massage effects to users of the massage chair at air massage pressure points. Due to the electro-mechanical movement to generate the compressed air, the air compressor can make a very unpleasant noise when it operates. In addition, the air valves' operation to control the air flow into and out of air cells are also noisy. Thus, a massage chair having a noise-reducing, device or system is desired for reducing noise generated from and/or made by these devices so as to make the massage environment more quiet and relaxing for users of the massage chair.

The disclosure overcomes one or more of the shortcomings of massage chairs, massage devices and apparatuses for massage chairs, and noise-reducing devices and systems. The Applicant is unaware of inventions or patents, taken either singly or in combination, which are seen to describe the disclosure as claimed. Additionally, the disclosure provides additional features for improving user experience including hammering devices, heating acupuncture devices, oxygen generation, voice recognition control of the massage chair, a remote control pouch or holder, and a wireless device pouch or holder having wireless charging functionality.

### SUMMARY OF THE DISCLOSURE

The disclosure is directed to a massage chair having a massage chair frame, a massage system that includes a plurality of air massage elements, and a noise-reducing (or noise-absorbing, noise-containing or noise-cancelling) enclosure device. Additionally, the disclosure is directed to additional features for improving user experience including hammering devices, heating acupuncture devices, oxygen generation, voice recognition control of the massage chair, a remote control pouch or holder, and a wireless device pouch or holder having wireless charging functionality.

The massage chair frame includes a first end, a second end, a seat or bottom body area portion, and a back body area portion extending upward from the seat or bottom body area portion toward the second end. Preferably, the massage chair frame also includes a thigh body area portion located between the seat or bottom body area portion and the first end, a head and neck body area portion extending upward from the back body area portion and located about the second end, and a lower leg body area portion located downward (or extending downward) from the thigh body area portion and located about the first end.

The massage system includes at least one air massage element, an air compressor or pump, and at least one air valve device for regulating air flow into and out of the at least one air massage element. Preferably, the massage system also comprises at least one air transport device and a power source. Also preferably, the at least one air massage element is a plurality of air massage elements, the at least one air valve device is a plurality of air valve devices, and the at least one air transport device is a plurality of air transport devices. The massage system is in operational communication with the massage chair frame such that the massage system provides massaging effects to a user positioned in the massage chair.

Each of the plurality of air massage elements is adapted for having air transported or pumped into it by the air compressor or pump and for having air withdrawn from it. The plurality of air massage elements may be positioned at predetermined massage locations on, about, or in the vicinity of the massage chair frame and/or may be moved or transported to desired massage locations by the user.

The air compressor or pump pumps air into the plurality of air massage elements such that massaging effects can be provided to the user at desired massage locations or pressure points.

The plurality of air valve devices regulate air flow into and out of the plurality of air massage elements.

The plurality of air transport devices transport air flow into and out of the plurality of air massage elements.

The power source provides power to the air compressor or pump, and may also be used to provide power to other components of the massage chair.

The noise-reducing (or noise-absorbing, noise-containing, or noise-cancelling) enclosure device includes an enclosure housing. The enclosure housing may comprise a layer of noise-reducing (or noise-absorbing, noise-containing, or noise-cancelling) material positioned inside. The enclosure housing encloses the air compressor or pump and the plurality of air valve devices during operation. The noise-reducing, enclosure device is preferably positioned in proximity of the massage chair frame.

The enclosure housing encloses the air compressor or pump and the plurality of air valve devices during operation such that noise generated from or made by the air compressor or pump and the plurality of air valve devices during operation is reduced, contained or eliminated.

Preferably, the noise-reducing (or noise-absorbing, noise-containing, or noise-cancelling) material is positioned inside the enclosure housing in such a way that it surrounds the air compressor or pump and the plurality of air valve devices such that noise generated from or made by the air compressor or pump and the plurality of air valve devices during operation is reduced, contained or eliminated.

### BRIEF DESCRIPTION OF THE DRAWINGS

FIG. 1 is a right side, perspective view of a massage chair according to the disclosure;

FIG. 2 is a perspective view of a massage chair frame for a massage chair according to the disclosure;

FIG. 3 is a front view of the massage chair frame of FIG. 2;

FIG. 4 is a right side view of the massage chair frame of FIG. 2;

FIG. 5 is a rear, partial perspective view of a massage chair frame and a massage system of a massage chair according to the disclosure;

FIG. 6 is a top, partial perspective view of a massage system and a noise-reducing, enclosure device of a massage chair according to the disclosure;

FIG. 7 is a top, perspective view of the noise-reducing, enclosure device of FIG. 6;

FIG. 8 is a bottom, perspective view of the noise-reducing, enclosure device of FIG. 6;

FIG. 9 is a perspective view of a securing nut of the noise-reducing, enclosure device of FIG. 6;

FIG. 10 is a top, perspective view of the massage system and the noise-reducing, enclosure device of FIG. 6, with a top of the noise-reducing, enclosure device being temporarily removed;

FIG. 11 is a partial cut-away, top, perspective view of the massage system and the noise-reducing, enclosure device of FIG. 6, with a top of the noise-reducing, enclosure device being temporarily removed;

FIG. 12 is a perspective view of noise-reducing material of the noise-reducing, enclosure device of FIG. 6;

FIG. 13A is a side, perspective view of an air massage element and an air transport device of a massage system of a massage chair according to the disclosure;

FIG. 13B is a perspective view of the air massage element and the air transport device of FIG. 13A;

FIG. 13C is an opposite side, perspective view of an air massage element and an air transport device of a massage system of a massage chair according to the disclosure;

FIG. 14 is an elevated, front view of a massage chair, showing multiple locations where air massage elements may be positioned;

FIG. 15 is an elevated, front view of a massage chair, showing multiple locations where hammer punching devices may be positioned;

FIG. 16 is a cross-sectional view of a hammer punching device according to the disclosure;

FIG. 17 is a perspective view of a hammer punching device according to the disclosure;

FIG. 18 is an exploded view of the chair with armrest removed according to the disclosure;

FIG. 19 is an armrest sliding feature according to another aspect of the disclosure;

FIG. 20 is a perspective view of a sliding wing, another mounting option for armrest sliding feature according to another aspect of the disclosure;

FIG. 20A is a side view of a slide rack;

FIG. 20B is a side view of the slide rack of FIG. 20A according to another aspect of the disclosure;

FIG. 20C is a side view of the slide rack of FIG. 20A according to another aspect of the disclosure;

FIG. 20D is a cross-sectional view of the slide rack of FIG. 20A;

FIG. 20E is a perspective view of a side rack and mounting mechanism with armrest sliding backward;

FIG. 20F is a back view of the slide rack and mounting mechanism;

FIG. 20G is a perspective view of an actuator;

FIG. 20H is a perspective view of another aspect of a slide rack and mounting mechanism according to the disclosure;

FIG. 20I is a cross-sectional view of a slide rack according to another aspect of the disclosure;

FIG. 20J is a back view of an armrest of another aspect of a supporting and mounting mechanism according to the disclosure;

FIG. 21 is a perspective view of an enclosure section for air valves according to the disclosure;

FIG. 22 is a perspective view of the enclosure section for air valves according to FIG. 21 with a top cover removed;

FIG. 23 is a perspective view of the enclosure section for air valves according to FIG. 21 with a top cover in front cover removed;

FIG. 24 is a left side view of the massage chair frame with an oxygen generator according to the disclosure;

FIG. 25 is a perspective view of an oxygen generator according to the disclosure;

FIG. 26 is an elevated front view of a massage chair having an oxygen distribution system according to the disclosure;

FIG. 27 is a perspective, right side view of a massage chair having a wireless device pouch or holder and a remote control pouch according to the disclosure;

FIG. 28 is a perspective view of a wireless charger configured to be implemented in the massage chair according to the disclosure;

FIG. 29A is a perspective, right side view of a massage chair having a media player holder according to the disclosure;

FIG. 29B is a perspective, right side view of a media player holder according to the disclosure;

FIG. 30A is a perspective, right side view of a massage chair having a media player holder according to another aspect of the disclosure;

FIG. 30B is a perspective, right side view of a media player holder according to another aspect of the disclosure;

FIG. 31A is a perspective, right side view of a massage chair having a shoulder airbag sliding according to the disclosure;

FIG. 31B is a perspective view of a shoulder supporter having an airbag and mounting mechanism according to the disclosure;

FIG. 31C is a perspective view of a slide rack and mounting mechanism according to the disclosure; and

FIG. 32 is an elevated front, environmental view of a user using a virtual reality (VR) device according to the disclosure.

It should be understood that the above-attached figures are not intended to limit the scope of the disclosure in any way.

### DETAILED DESCRIPTION OF THE PREFERRED EMBODIMENTS

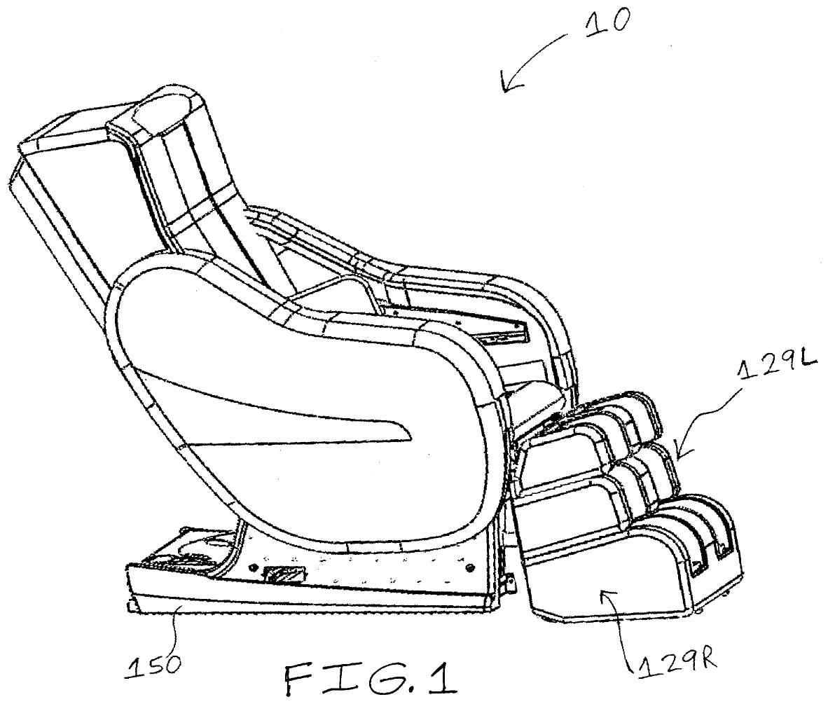

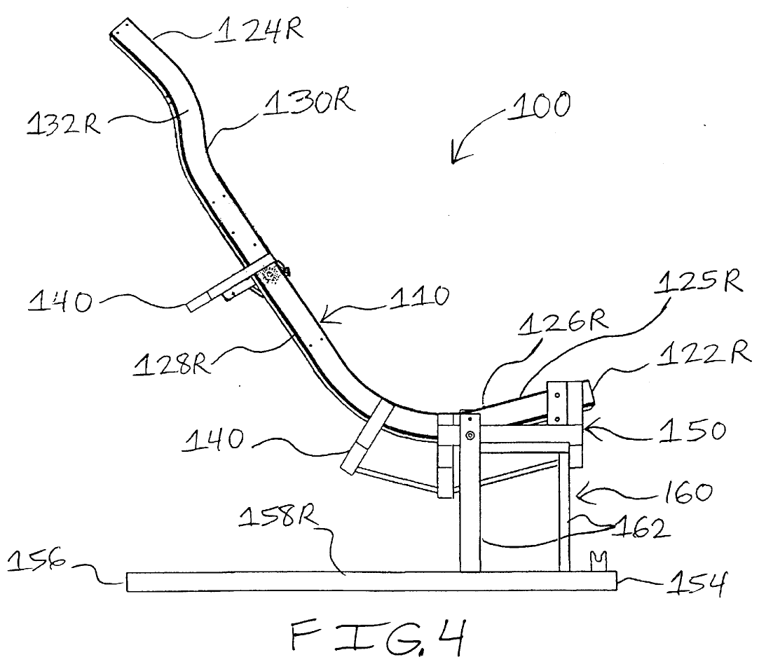

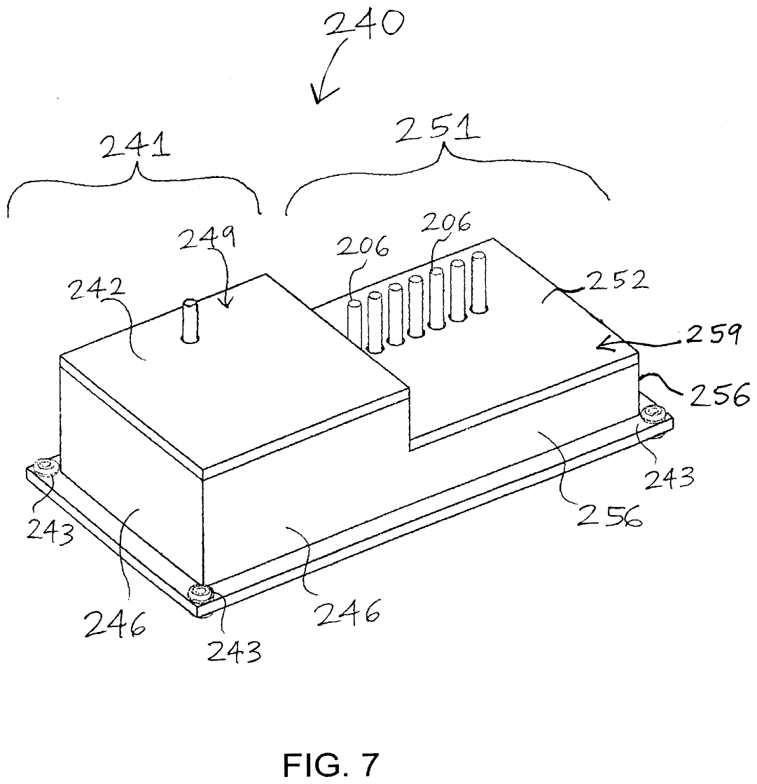

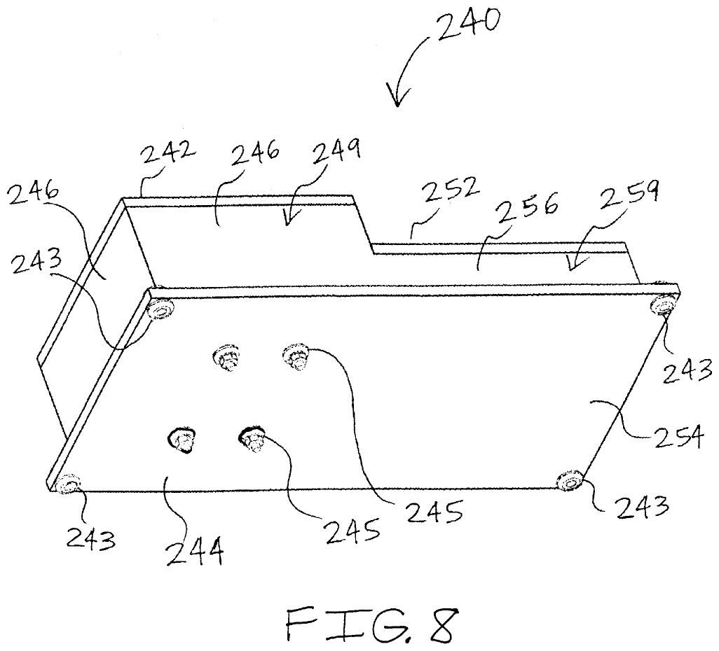

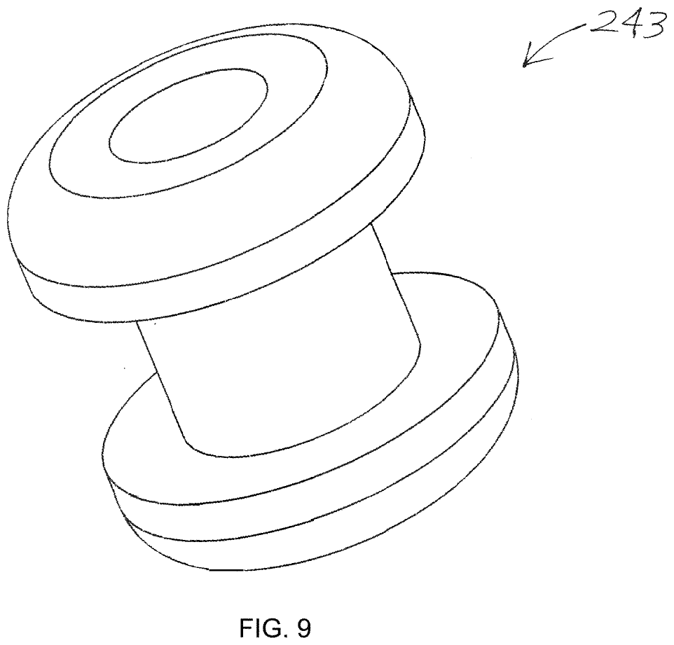

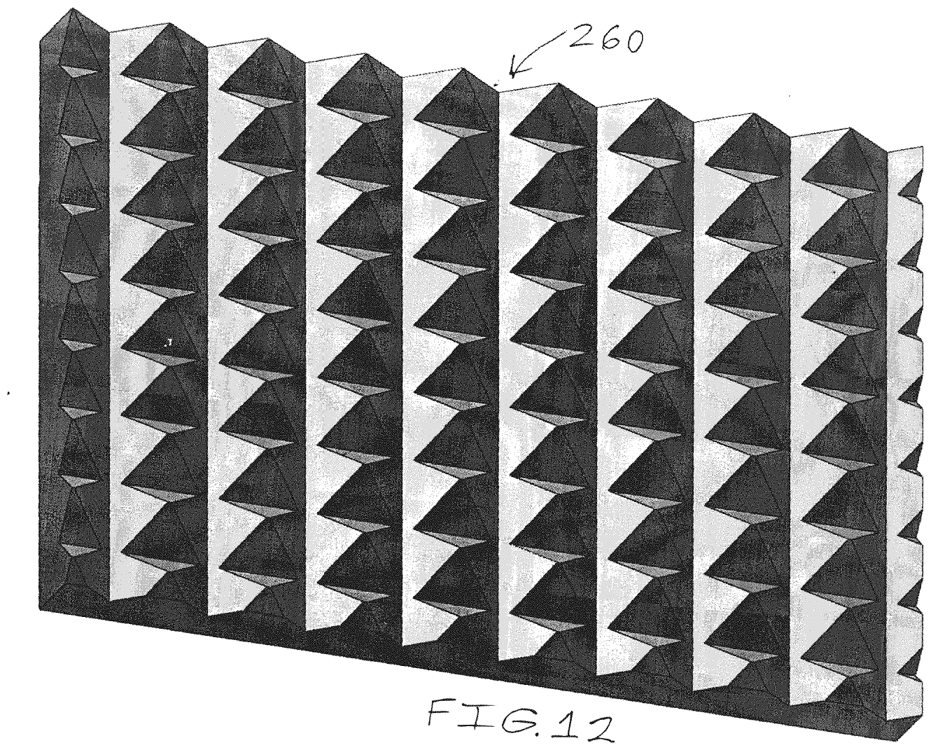

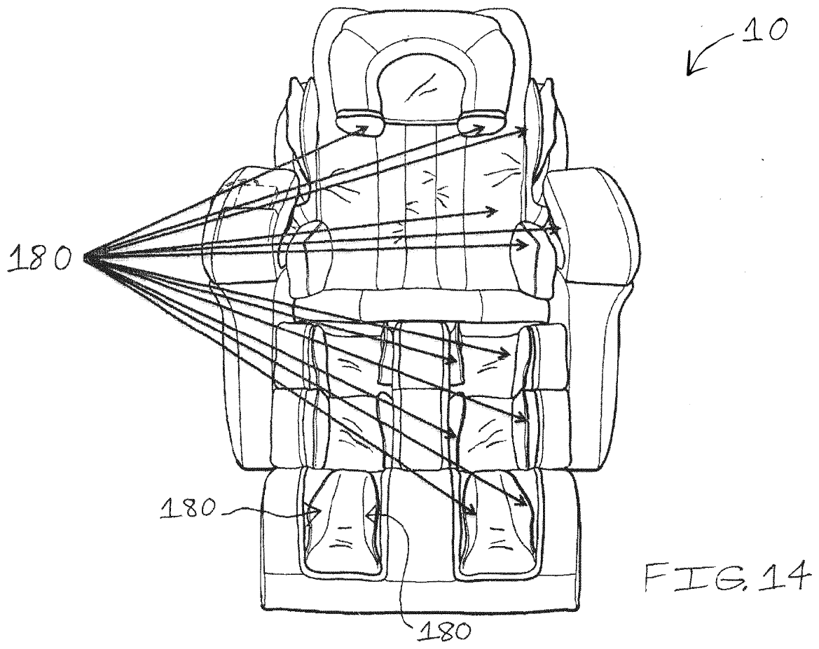

Referring to FIGS. 1-14, the disclosure is directed to a massage chair 10 having a massage chair frame 110, a massage system 170 that includes a plurality of air massage elements 180, and a noise-reducing (or noise-absorbing, noise-containing or noise-cancelling) enclosure device 230.

The massage chair frame 110 may include a first end, a second end, a seat or bottom body area portion, and a back body area portion extending upward from the seat or bottom body area portion toward the second end. Preferably, the massage chair frame 110 also includes a thigh body area portion located between the seat or bottom body area portion and the first end, a head and neck body area portion extending upward from the back body area portion and located about the second end, and a lower leg body area portion located downward (or extending downward) from the thigh body area portion and located about the first end. In general, the massage chair frame 110 may be any massage chair frame known to one of ordinary skill in the art that comprises at least a seat or bottom body area portion and a back body area portion.

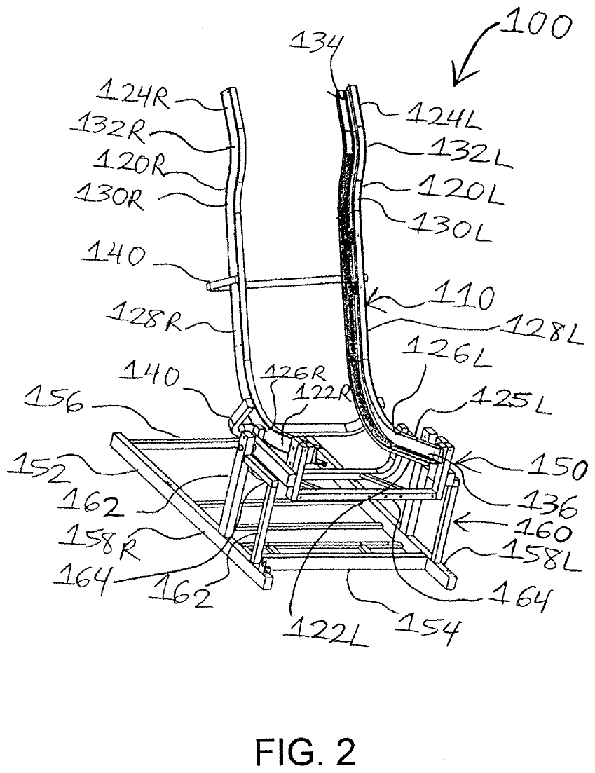

As a non-limiting example and as best shown in FIGS. 2-4, the massage chair frame 110 includes a pair of opposing guide rails 120R, 120L, a plurality of guide rails stabilizing bars 140, and a base stand 150. The guide rails 120R, 120L are secured to the base stand 150, and are positioned generally above the base stand 150. The base stand 150 supports the weight of the guide rails 120R, 120L and, preferably, also the weight of a user (not shown) of the massage chair.

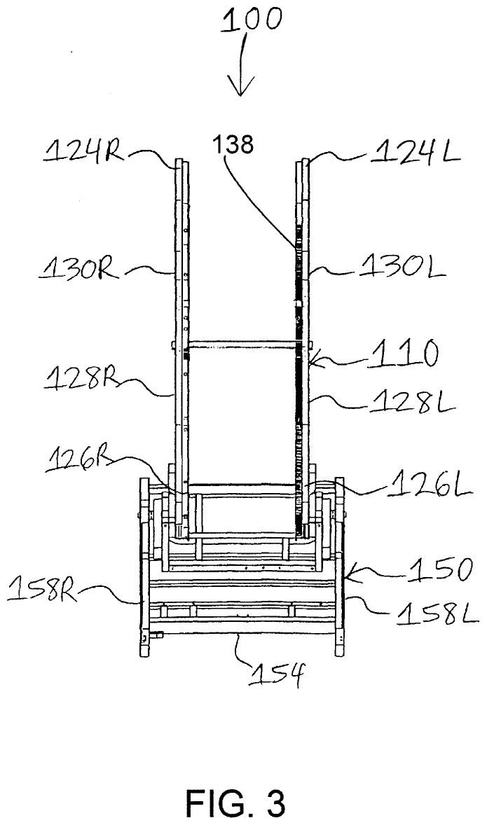

Preferably, the guide rails 120R, 120L are substantially similar or mirror images of one another. Each of the guide rails 120R, 120L includes a first end 122R, 122L, a second end 124R, 124L, a thigh body area portion 125R, 125L located adjacent the first end 122R, 122L, a seat or bottom body area portion 126R, 126L located adjacent the thigh body area portion 125R, 125L and away from the first end 122R, 122L, a back body area portion 128R, 128L extending upward from the bottom body area portion 126R, 126L, a head and neck body area portion 130R, 130L extending upward from the back body area portion 128R, 128L and located about the second end 124R, 124L, an outer side 132R, 132L, an inner side 134, and a guide channel 136 extending from the thigh body area portion 125R, 125L to the back body area portion 128R, 128L, preferably to the head and neck body area portion 130R, 130L, and running along the inner side 134 of the guide rail 120R, 120L. The guide channel 136 may include gear teeth 138 for engaging with at least one gear member from a mechanical massage device (not shown) when the mechanical massage device moves upward and downward in a generally vertical direction from the first end 122R, 122L toward the second end 124R, 124L of the guide rail 120R, 120L and vice versa, respectively. Preferably, each of the guide rails 120R, 120L has a generally “L-shaped” configuration. In this configuration, the lower portion of the “L” includes the thigh body area portion 125R, 125L and bottom body area portion 126R, 126L, and the upper portion of the “L” includes the back body area portion 128R, 128L and head and neck body area portion 130R, 130L. As best shown in FIGS. 1 and 3, more preferably, each of the guide rails 120R, 120L has a reclining “L-shaped” configuration.

Preferably, as shown in FIG. 1, the lower leg body area portion 129R, 129L is a further extension of the massage chair frame 110, or is an add-on device that is secured or attached about the first end 122R, 122L of the guide rails 120R, 120L of the massage chair frame 110. Preferably, the lower leg body area portion 129R, 129L is located downward (or extending downward) from the thigh body area portion 125R, 125L and located downward (or extending downward) from the first end 122R, 122L.

The plurality of guide rails stabilizing bars 140 help to stabilize the positioning of the guide rails 120R, 120L relative to one another. Each of the guide rails stabilizing bars 140 has a first end 142, a second end 144, and a body portion 146 extending from the first end 142 to the second end 144. Preferably, each of the guide rails stabilizing bars 140 has a generally “U-shaped” configuration. The guide rails stabilizing bars 140 are secured at predetermined locations along the outer sides 132R, 132L of the guide rails 120R, 120L.

The base stand 150 includes a base 152 and a guide rails support structure 160. The base 152 includes a first or front end 154, a second or rear end 156, and a pair of opposing sides 158R, 158L. The guide rails support structure 160 is secured about the front end 154 of the base 152, and is positioned above the base 152. The guide rails support structure 160 includes a plurality of vertical bars or vertical members 162 and a plurality of horizontal bars or members 164. The plurality of vertical bars 162 extend upward from the pair of opposing sides 158R, 158L of the base 152, and, along with the plurality of horizontal bars 164, form a support frame with a “square-shaped” or “rectangular-shaped” box configuration.

Since the base stand 150 supports the weight of the guide rails 120R, 120L and user of the massage chair, the base stand 150 is preferably made or manufactured of a strong material, such as, but not limited to, steel, metal, wood, hard plastic, any material or combination of materials known to one of ordinary skill in the art, and any combination thereof. Also, each of the guide rails 120R, 120L and plurality of guide rails stabilizing bars 140 may be made or manufactured of steel, metal, wood, plastic, any material or combination of materials known to one of ordinary skill in the art, and any combination thereof.

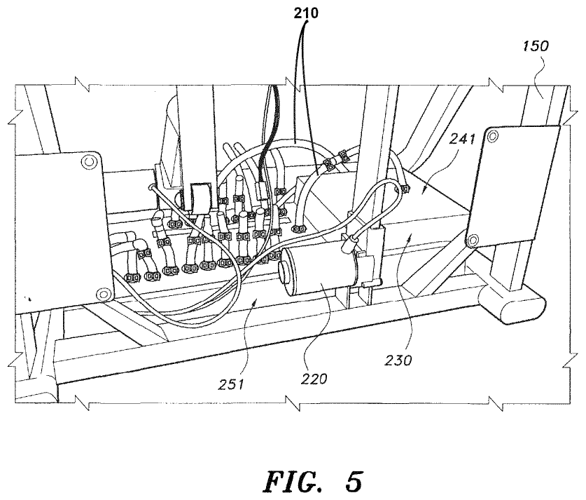

As best shown in FIGS. 5, 6, 14, and 15, the massage system 170 includes at least one air massage element 180, an air compressor or pump 190, and at least one air valve device 200 for regulating air flow into and out of the at least one air massage element 180. Preferably, the massage system 170 also includes at least one air transport device 210 and an actuator 220. Also preferably, the at least one air massage element 180 is a plurality of air massage elements 180, the at least one air valve device 200 is a plurality of air valve devices 200, and the at least one air transport device 210 is a plurality of air transport devices 210. The massage system 170 is in operational communication with the massage chair frame 110 such that the massage system 170 provides massaging effects to a user (not shown) positioned in the massage chair 10.

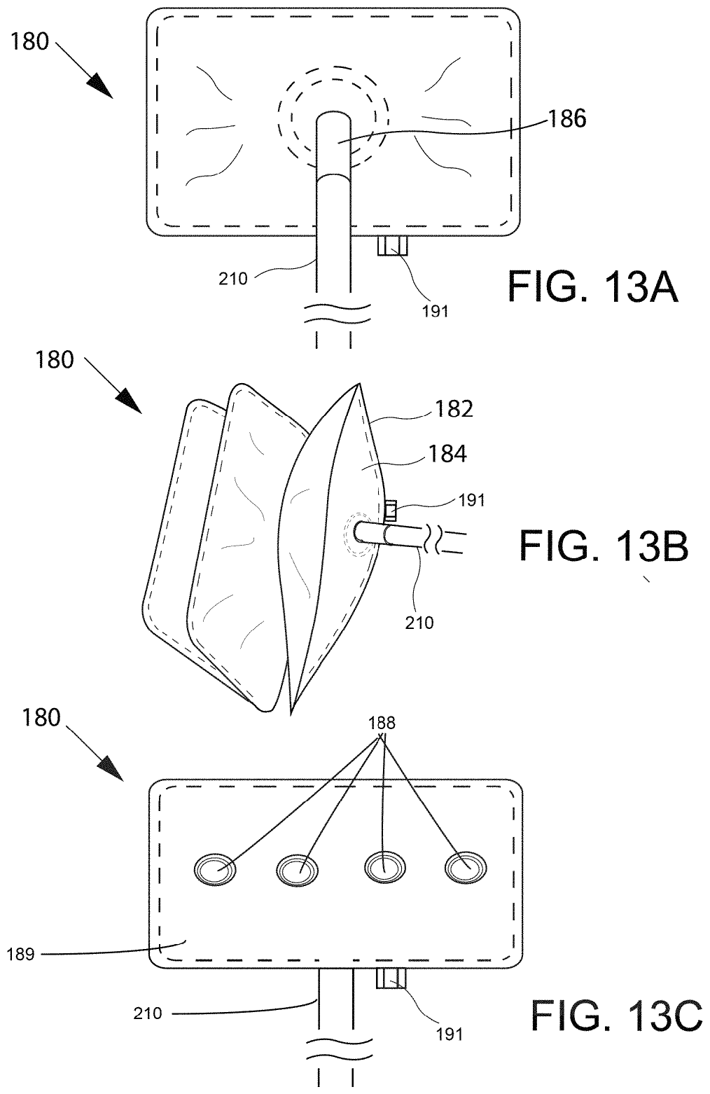

Each of the plurality of air massage elements 180 is adapted for having air transported or pumped into it by the air compressor or pump 190 and for having air withdrawn from it. The plurality of air massage elements 180 may be positioned at predetermined massage locations on, about, or in the vicinity of the massage chair frame 110 and/or may be moved or transported to desired massage locations by the user. The plurality of air massage elements 180 may be a plurality of air massage cells (such as, but not limited to, air cell 180 shown in FIGS. 13A, 13B, and 13C), a plurality of air massage bags (such as, but not limited to, air bags), any air massage element(s) known to one of ordinary skill in the art, and any combination thereof. The air cell 180 includes an inflatable or expandable air cell housing 182, an air cell chamber 184 defined by the air cell housing 182, and an air cell inlet and outlet 186. The air that is contained in and/or pumped into and out of the plurality of air massage elements 180 may be air, a liquid, a gel, any air(s) known to one of ordinary skill in the art, and any combination thereof.

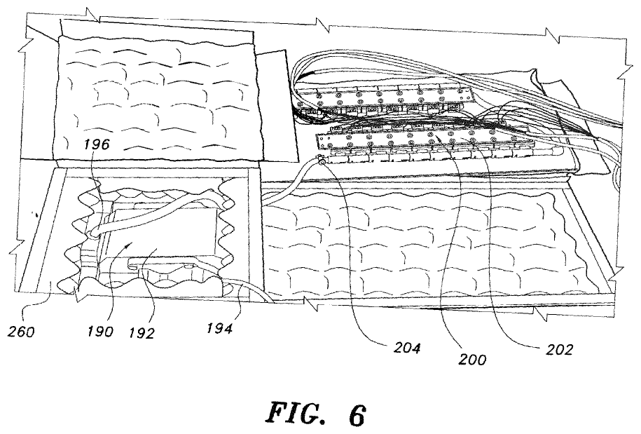

The air compressor or pump 190 pumps air into the plurality of air massage elements 180 such that massaging effects can be provided to the user at desired massage locations or pressure points. The air compressor or pump 190 comprises an air pump body 192, an air pump power source 194, and an air pump outlet 196. The air compressor or pump 190 (such as the non-limiting example shown in FIGS. 6, 10 and 11) may be any air compressor or pump known to one of ordinary skill in the art that is able to pump air, a liquid, a gel, any air(s) known to one of ordinary skill in the art, and any combination thereof into the plurality of air massage elements 180.

The plurality of air valve devices 200 regulate air flow into and out of the plurality of air massage elements 180. Each of the air valve device 200 comprises an air valve body 202, an air valve inlet 204, and an air valve outlet 206. Each of the plurality of air valve devices 200 (such as the non-limiting example shown in FIGS. 6, 10 and 11) may be any air valve device known to one of ordinary skill in the art that is able to regulate air flow into and out of the corresponding air massage element(s) 180.

The plurality of air transport devices 210 transport air flow into and out of the plurality of air massage elements 180. Each of the plurality of air transport devices 210 (such as the non-limiting example shown in FIGS. 13A, 13B, and 13C) may be any air transport device known to one of ordinary skill in the art that is able to transport air flow into and out of the corresponding air massage element(s) 180.

The power source 194 provides power to the air compressor or pump 190, and may also be used to provide power to other components of the massage chair 100. The power source 194 may be a battery, a connector or cord for plugging into a power outlet (such as, but not limited to, a detachable DC power supply cord), a plug for receiving power or electricity, any power source known to one of ordinary skill in the art, and any combination thereof.

As best shown in FIGS. 5-12, the noise-reducing (or noise-absorbing, noise-containing, or noise-cancelling) enclosure device 230 includes an enclosure housing 240 and noise-reducing (or noise-absorbing, noise-containing, or noise-cancelling) material 260 positioned inside the enclosure housing 240. The enclosure housing 240 encloses the air compressor or pump 190 and the plurality of air valve devices 200 during operation. The noise-reducing enclosure device 230 is preferably positioned in proximity of the massage chair frame 110. As a non-limiting example shown in FIGS. 1 and 5, the noise-reducing, enclosure device 230 is positioned below the seat or bottom body area portion 126R, 126L of the massage chair frame 110.

As a non-limiting example and as best shown in FIGS. 6-11, the enclosure housing 240 has a first section 241 that includes a top 242, a bottom 244, a plurality of sides 246, an inner surface 248, and an outer surface 249, and a second section 251 that includes a top 252, a bottom 254, a plurality of sides 256, an inner surface 258, and an outer surface 259. Preferably, the inner surfaces 248, 258 and noise-reducing (or noise-absorbing, noise-containing, or noise-cancelling) material 260 help to form noise-reducing (or noise-absorbing, noise-containing, or noise-cancelling) walls for the enclosure housing 240. The first section 241 of the enclosure housing 240 encloses (partially, substantially, or fully encloses) the air compressor or pump 190 while the second section 251 of the enclosure housing 240 encloses (partially, substantially, or fully encloses) the plurality of air valve devices 200 such that noise generated from or made by the air compressor or pump 190 and the plurality of air valve devices 200 during operation is reduced, contained, or eliminated. The enclosure housing 240 also has a plurality of foot elements 243 positioned at predetermined locations on the periphery of the enclosure housing 240, and a plurality of screw and nut elements 245 positioned at predetermined locations.

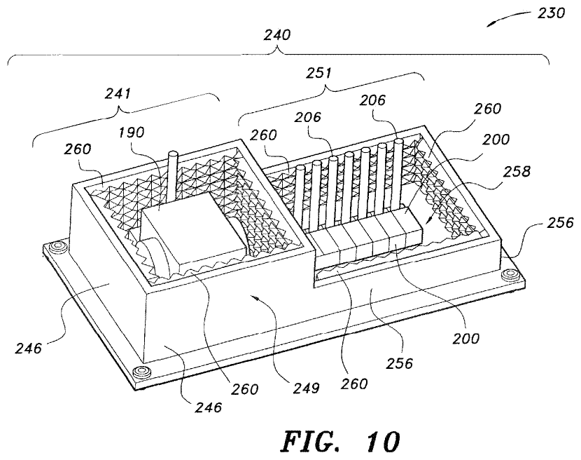

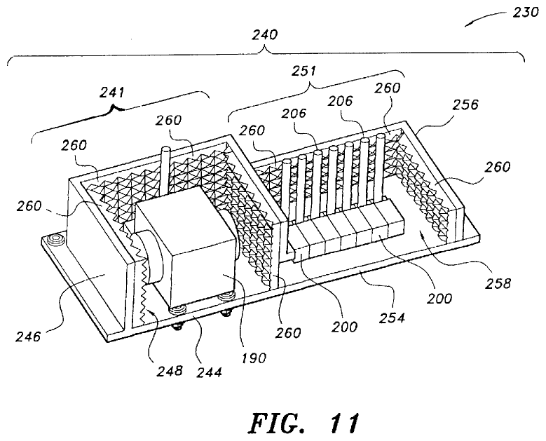

Preferably, the noise-reducing (or noise-absorbing, noise-containing, or noise-cancelling) material 260 is positioned inside the enclosure housing 240 in such a way that it surrounds the air compressor or pump 190 and the plurality of air valve devices 200 such that noise generated from or made by the air compressor or pump 190 and the plurality of air valve devices 200 during operation is reduced, contained or eliminated. Preferably, as shown in FIGS. 10 and 11, all of the inner surfaces 248, 258 of the enclosure housing 240 are covered by the noise-reducing material 260. Preferably, all of the electro-mechanical components positioned inside the enclosure housing 240 are fully surrounded by the noise-reducing material 260. The noise-reducing material 260 may be foam, noise-reducing foam, noise-absorbing foam, noise-containing foam, noise-cancelling foam, any noise-reducing material known to one of ordinary skill in the art, any noise-absorbing material known to one of ordinary skill in the art, any noise-containing material known to one of ordinary skill in the art, any noise-cancelling material known to one of ordinary skill in the art, and any combination thereof.

When in use or in operation, the user (not shown) may activate the massage system 170 of the massage chair 100 by or via pushing, touching area of touchscreen remote controller 11, using voice command for use on or with, using a mechanical or remote control for use on or with, or any other activation method known to one of ordinary skill in the art, an activation, start, control or command button, touch area, box or panel, or any other activation method or element known to one of ordinary skill in the art. Preferably, the user is able to control the massage producing-effects of the plurality of air massage elements 180 such that the plurality of air massage elements 180 provide massage producing-effects to a desired body part area(s), such as the thighs, bottom, lower back, upper back, head and neck, and/or lower leg of the user so that desired body part area(s) of the user can receive massage effects or benefits from the plurality of air massage elements 180 when desired.

Additionally, FIG. 13A, FIG. 13B, and FIG. 13C further illustrate that the plurality of air massage elements 180 may include heating functionality. In this regard, as illustrated in FIG. 13C, the plurality of air massage elements 180 may include heated acupunctures 188. The heated acupunctures 188 may be located on an air cell front side 189. The heated acupunctures 188 may be located in other locations on or within the massage chair 10 as well. The heated acupunctures 188 may include a heating device to raise a temperature of the heated acupunctures 188. The heated acupunctures 188 may further include a power connector 191 to provide power to the heated acupunctures 188 for operating the heating device.

The heated acupunctures 188 may further include a circuit, a touch screen remote controller 11, or the like to control or vary a temperature of the heated acupunctures 188 in response to user input or a temperature sensor. In one aspect, the user input may be input to an input device. The input device may include switches, buttons, a touch sensitive screen of a touchscreen remote controller, and the like. The user input may also include voice activation or voice control utilizing voice recognition software.

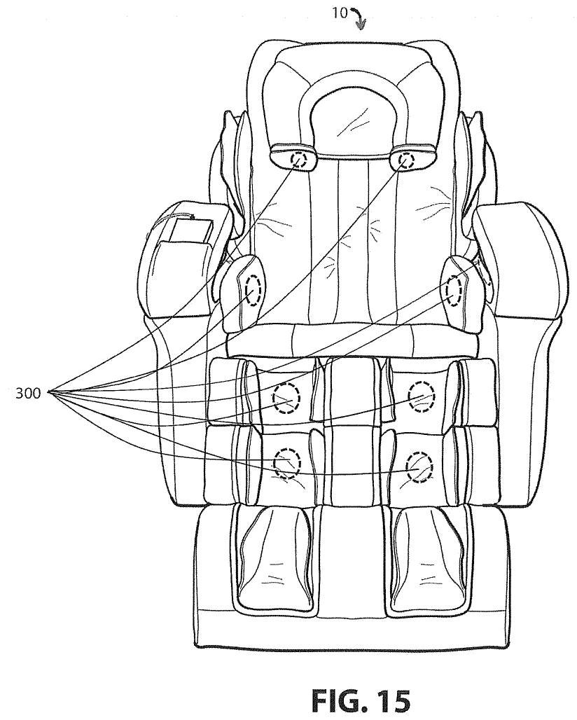

FIG. 15 is an elevated, front view of a massage chair, showing multiple locations where hammer punching devices may be positioned.

In particular, FIG. 15 illustrates the massage chair 10 having a plurality of hammer punching devices 300 located at various locations in the massage chair 10. In one aspect, one or more of the plurality of hammer punching devices 300 may be located adjacent headrest. In one aspect, one or more of the plurality of hammer punching devices 300 may be located adjacent a thigh area. In one aspect, one or more of the plurality of hammer punching devices 300 may be located adjacent an armrest area. In one aspect, one or more of the plurality of hammer punching devices 300 may be located adjacent a lower leg area. Other locations for the plurality of hammer punching devices 300 are contemplated as well.

In particular, the hammer punching devices 300 may provide a punching massage effect to a user positioned in the massage chair 10. In operation, the hammer punching devices 300 may provide a rapid movement and associated application of repeated pressure and/or impact to a user at various locations of the user's body. In this regard, the hammer punching devices 300 may provide a massaging affect to the user as they are sitting in the massage chair 10.

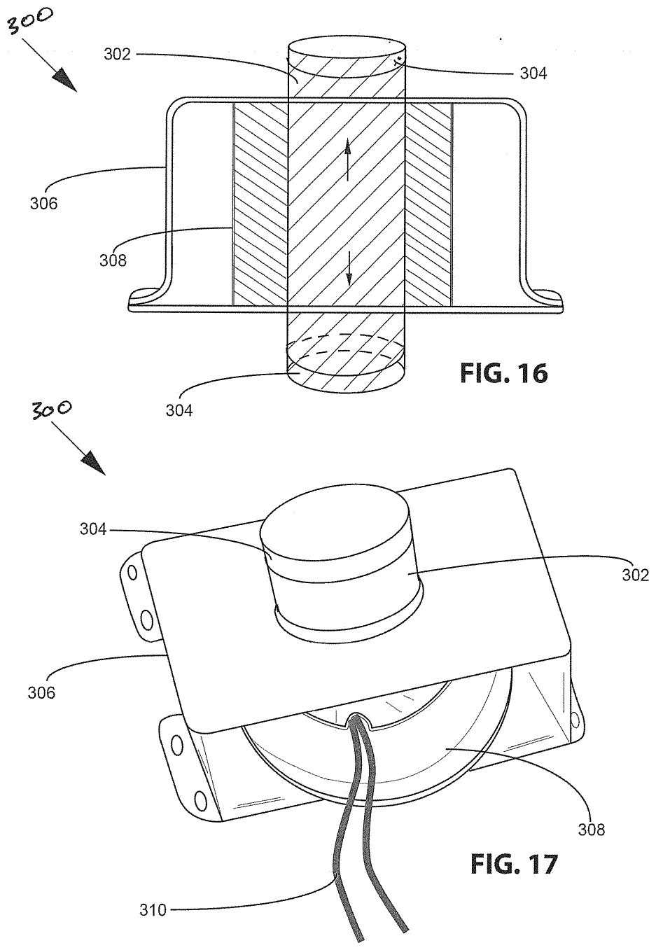

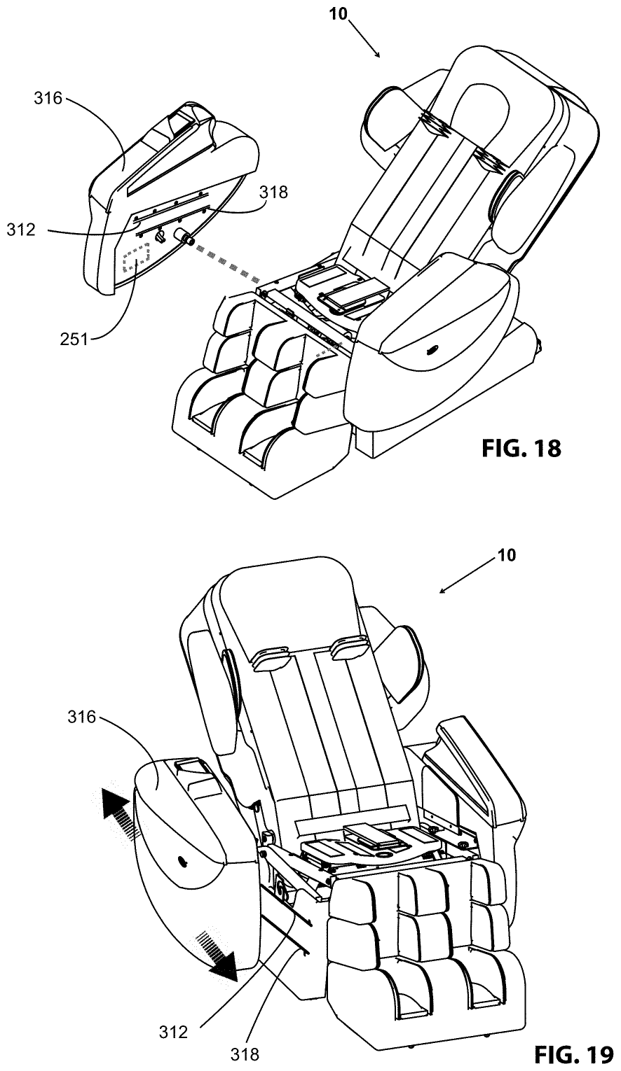

FIG. 16 illustrates a cross-sectional view of a punching device according to the disclosure; and FIG. 17 illustrates a perspective view of a punching device according to the disclosure.

In particular, FIG. 16 and FIG. 17 illustrate details of one of the hammer punching devices 300. The hammer punching device 300 may include a punching cylinder 302 configured to move with respect to a housing 306 as indicated by the arrows in FIG. 16. One or both ends of the punching cylinder 302 may include a soft layer 304. The soft layer 304 may include leather, plastic, rubber, a synthetic material, or the like. In one aspect, the soft layer 304 may soften the impact of the punching cylinder 302 on a user.

The hammer punching device 300 may further include a coil 308 to move the punching cylinder 302 as indicated by the arrows when provided with power from a power supply 310. The power supply 310 may be an alternating current (AC) power supply or a direct current (DC) power supply. The hammer punching device 300 may further include a circuit, controller, microprocessor, or the like to control the hammer punching device 300. In particular, the hammer punching device 300 may be controlled such that the punching cylinder 302 has a particular range of motion, a frequency of movement, and a set amount of force. In one aspect, the hammer punching device 300 may be controlled by user input. In one aspect, the user input may be input to an input device. The input device may include switches, buttons, a touch sensitive screen of a touchscreen remote controller, and the like. In one aspect, user input may also include voice activation or voice control utilizing voice recognition software.

The housing 306 may house the coil 308 and it may partially house the punching cylinder 302. The housing 306 may further include a noise reducing material 206 consistent with the remaining disclosure.

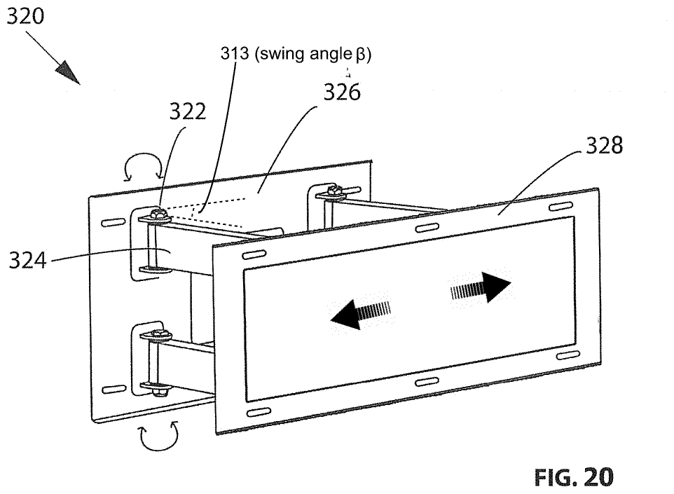

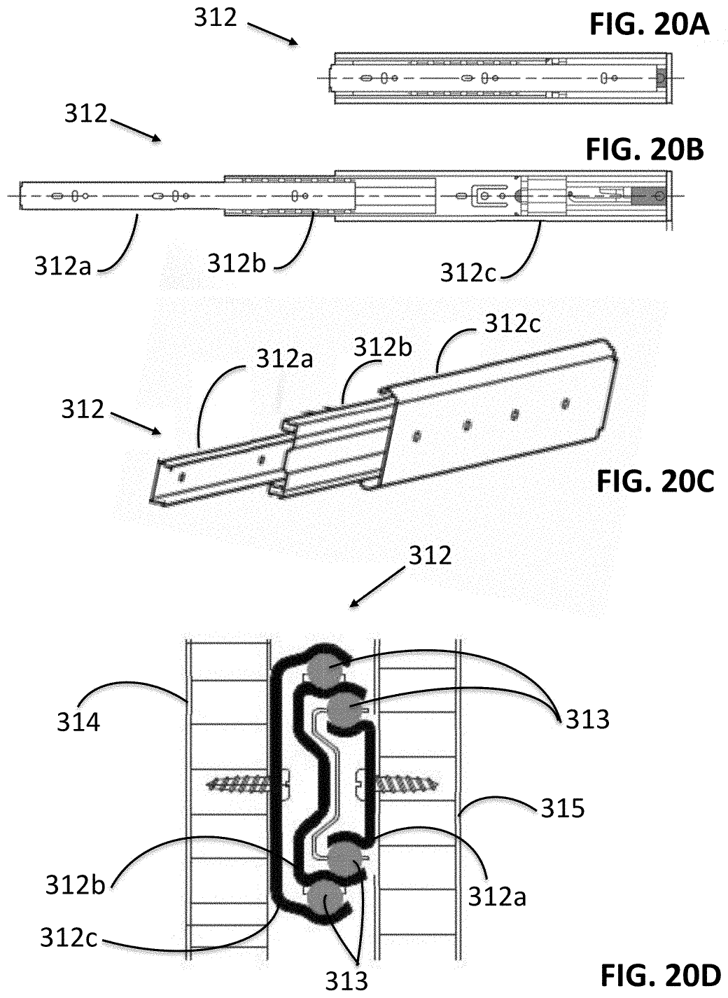

FIG. 18 is an exploded view of the chair with armrest removed according to the disclosure; and FIG. 19 illustrates an armrest sliding feature according to another aspect of the disclosure.

In particular, FIG. 18 and FIG. 19 illustrate a slide rack that may allow the armrest 316 to slide forward or backward for easy accessing the chair 10 wherein the sliding rack may be mounted to a side of a base stand of the massage chair 10. Additionally, the slide rack may include at least a locking mechanism 329 to lock the armrest 316 at one position.

In particular, an armrest 316 may include a second section 251, a first slide rack 312, and a second slide rack 318. In one aspect, the first slide rack 312 and the second slide rack 318 may cooperate to mechanically fasten the armrest 316 to the massage chair 10. Additionally, the first slide rack 312 and the second slide rack 318 may cooperate to mounting mechanisms 325 to allow the armrest 316 to move toward the front of the massage chair 10 as well as, alternatively, move toward the rear of the massage chair 10. In this regard, the armrest 316 may be positioned at any desired location consistent with the desires of the user. Moreover, the armrest 316 may be removed for shipping as well as delivery.

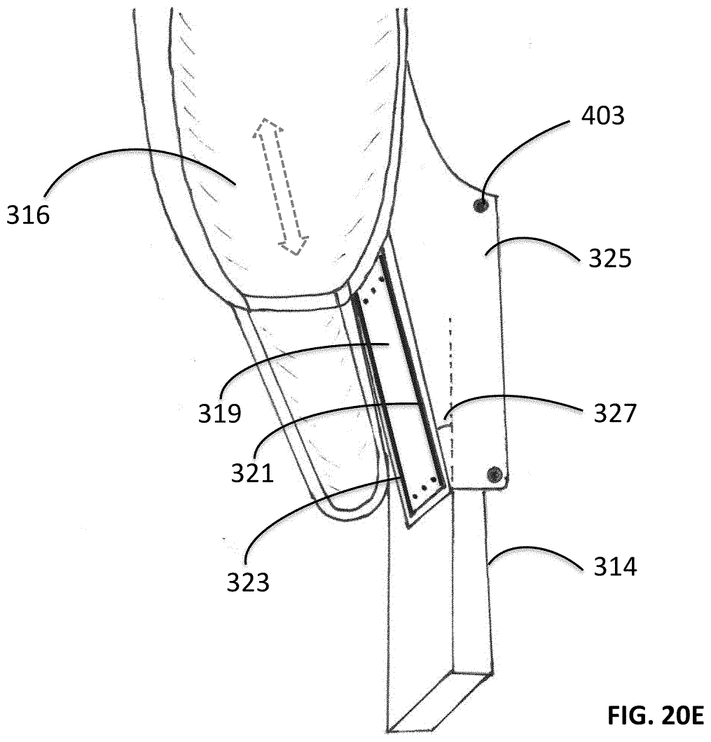

FIG. 20 is a perspective view of a sliding wing, another mounting option for armrest sliding feature according to another aspect of the disclosure.

In particular, FIG. 20 illustrates a support structure 320 for supporting an armrest 316. The support structure 320 may have a plurality of arms 324 that are pivotally connected to a base structure 326. In particular, the arms 324 may pivot as indicated by the arrows on a pivot joint 322 connected to the base structure 326. The arms 324 may pivot with an angle 313 (β), angle 313 is less than or equal to 180 degree. This allows an armrest connection portion 328 of the support structure 320 to be moved to any desired position. In one aspect, the support structure 320 may support the armrest 316 on the armrest connection portion 328. In particular, the base structure 326 may be connected to a side of the massage chair 10 and the armrest 316 may be connected to the armrest connection portion 328. With this configuration, the armrest 316 being connected to the armrest connection portion 328 may pivot and translate in a forward and, alternatively, backward motion to be placed in an infinite number of positions as desired by the user of the massage chair 10.

FIG. 20A, FIG. 20B, FIG. 20C, and FIG. 20D illustrate a slide rack 312 (or 318).

In particular, FIG. 20B and FIG. 20C illustrate a slide rack 312 that may include three extension sections 312a, 312b, and 312c.

In particular, FIG. 20D is cross-sectional view of slide rack 312. To reduce the friction, the slide rack may have multiple bearings, preferably ball bearings. Extension sections 312a, 312b, and 312c are engaged to each other and each comprises a curve to receive a bearing. One section is mounted to the massage chair frame while the other is mounted to a mounting mechanism or armrest. Slide rack 312 and bearings 313 are preferably made by a hard material, preferably by metal or a metal material.

FIG. 20E, FIG. 20F, and FIG. 20H illustrate other aspect of a slide rack.

In particular, slide rack 319 may have two parallel slide rails, a first (or top) slide rail and a second (or bottom) slide rail. Mounting mechanism 325 may have multiple mounting screws 403 to mount the mounting mechanism 325 to the massage chair frame 314. Mounting mechanism 325 is designed to have an offset angle 327 (angle α) to massage chair frame to avoid the collision between armrest and backrest during sliding or swinging. Angle 327 is greater than zero (or 0) degree but smaller than 90 degrees. Mounting mechanism 325 may be a single part or many parts assembled together.

In particular, FIG. 20F illustrates a mounting mechanism 325 and slide rack 319.

The mounting mechanism 325 may have a locking mechanism 329, a mounting tab 401, and mounting holes. The locking mechanism 329 may have a knob, a spring, and a pointer to engage with other locking feature (e.g., locking holes 405 as shown on FIG. 20H) to lock the armrest at one position (not shown). To slide the armrest, user may pull or push the locking mechanism 329 to release the locking.

The slide rack 319 may have an actuator 315, first (or top) slide rail 321 and second (or bottom) slide rail 323. The slide rack 319 and mounting mechanism 325 are made by a hard material, preferably by metal or a metal material.

FIG. 20G illustrates a side view of an actuator. The driver 331 of actuator 315 may be an electric motor or an air pump. The piston rod 333 moves according to the gear of electric motor or air pressure of an air pump. The actuator has two ends, first end mounted to the massage chair frame and second end mounted to the armrest or sliding rack or mounting mechanism. Power for the actuator 315 may be supplied by the massage chair 10 via a power cord. User can activate the actuator 315 via a touch button on touchscreen controller. A touch button takes input from user then sends a signal or code to the motherboard of massage chair to move the piston rod 333 forward or backward. Moving of piston rod 333 causes the armrest sliding forward or backward. The touchscreen controller and motherboard of massage chair 10 also comprise a software code to control the speed of the actuator 315. Other aspect, the armrest 316 may be designed and configured to have a control panel to activate the actuator 315. The armrest is removable for easy installation, therefore, there is a cable that connects the control panel to the motherboard or the actuator 315 that has at least two sections joined together via a connector. There is an open hole on the armrest to install a connector or to run a cable through.

FIG. 20H and FIG. 20J illustrate another aspect of slide rack 319 and mounting mechanism. In particular, the first (or top) slide rail 321, and second (or bottom) slide rail 323 may include a curve 317 so that the user can pull the armrest 316 out then slides just like sliding a side door of a van. Pulling the armrest 316 out then sliding will also avoid the collision of the armrest to the backrest. Other option is that armrest 316 may include a supporting mechanism (not shown) so that user still be able to pull the armrest 316 out then slide. In this case, the supporting mechanism may include multiple supporting arms 407 that engage with the guide rails 321 and 323. The supporting arms 407 may include bearing 313 to reduce the friction during sliding and a spring 409 to reduce the pulling (or pushing) force. When user pulls (or pushes) the armrest, the supporting arms 407 may move in and out from the armrest 316. The mounting mechanism 325 may have plurality locking holes 405, several mounting screws 403. The slide rail 321 is parallel with slide rail 323.

FIG. 20I illustrates a cross-sectional view of slide rack 319. In particular, slide rack 319 may have first (or top) slide rail 321, and second (or bottom) slide rail 323. Slide rack 319 may also have first portion 319a engages with second portion 319b. First portion 319a and second portion 319b may be configured to receive bearings 313 to reduce the friction during sliding. Preferably, bearing 313 is ball bearing. The first portion 319a may be mounted to the armrest or mounting mechanism while the second portion 319b may be mounted to the massage chair frame 314.

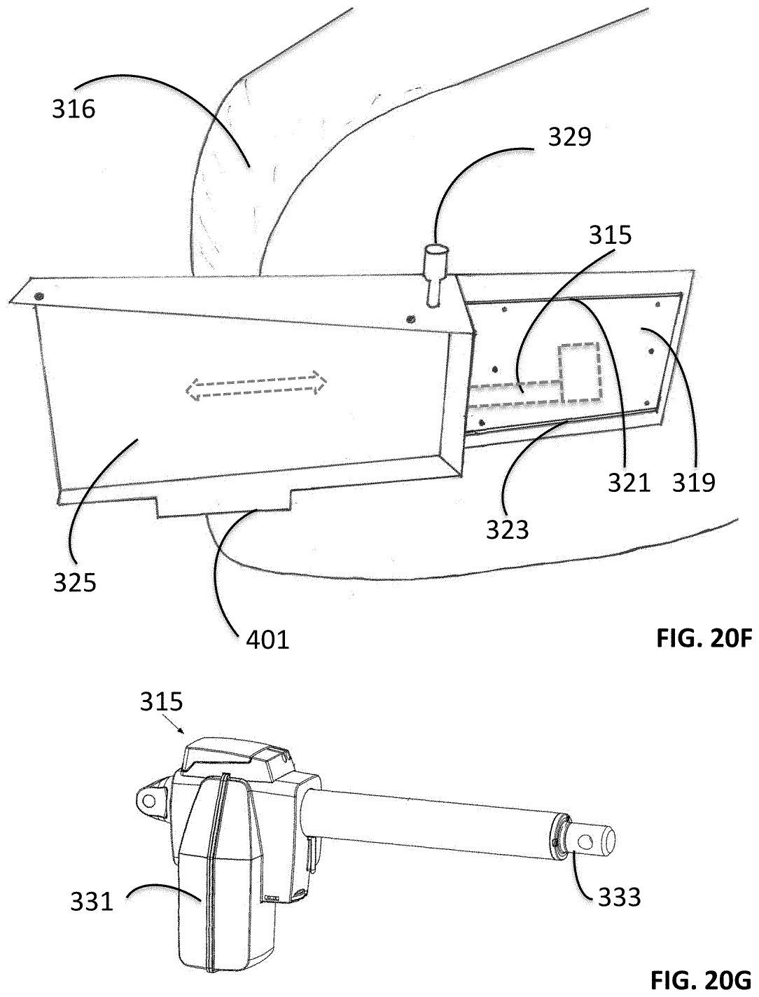

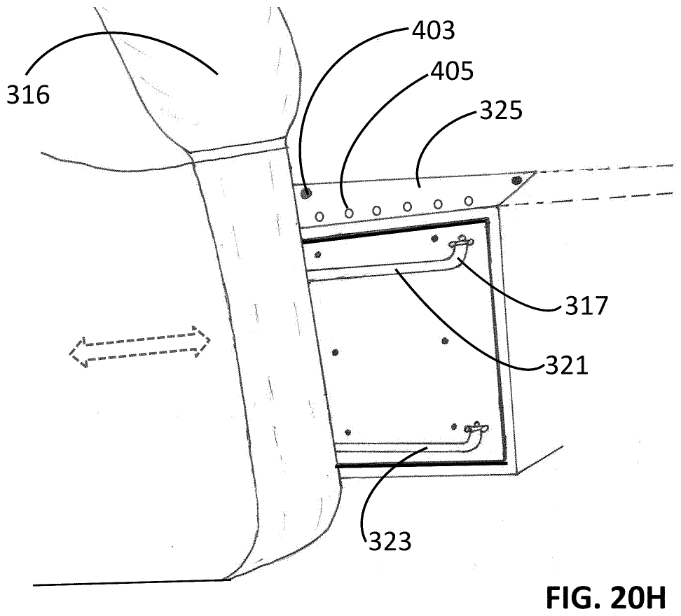

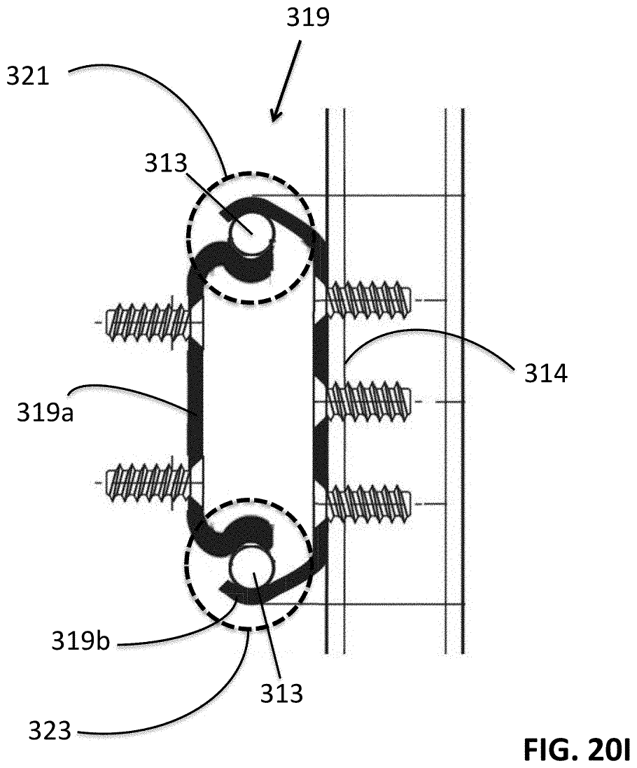

FIG. 21 illustrates a perspective view of an enclosure section for air valves according to the disclosure; FIG. 22 illustrates a perspective view of the enclosure section for air valves according to FIG. 21 with a top cover removed; and FIG. 23 illustrates a perspective view of the enclosure section for air valves according to FIG. 21 with a top cover in front cover removed.

In particular, FIG. 21, FIG. 22, and FIG. 23, illustrate the second section 251 of an enclosure housing that encloses (partially, substantially, or fully encloses) the plurality of air valve devices 200 such that noise generated from or made by the plurality of air valve devices 200 during operation is reduced, contained, or eliminated. The second section 251 may also have a plurality of foot elements 243 positioned at predetermined locations on the periphery of the second section 251. Additionally, the second section 251 may be configured to allow the air valve outlets 206 to extend from the second section 251 for connection to the plurality of air massage elements 180.

The second section 251 may include a top 252, a plurality of sides 246, a plurality of sides 256, an inner surface 258, and an outer surface. Preferably, the inner surfaces 258 and noise-reducing (or noise-absorbing, noise-containing, or noise-cancelling) material 260 form noise-reducing (or noise-absorbing, noise-containing, or noise-cancelling) walls for the second section 251. The second section 251 encloses (partially, substantially, or fully encloses) the plurality of air valve devices 200.

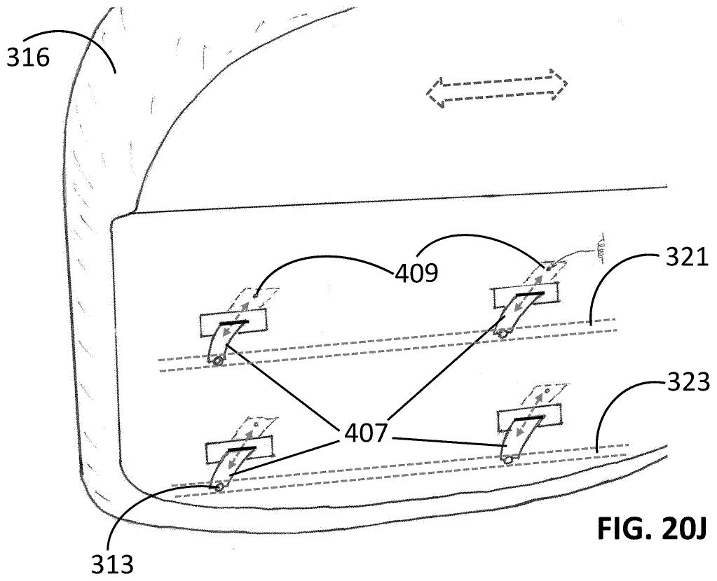

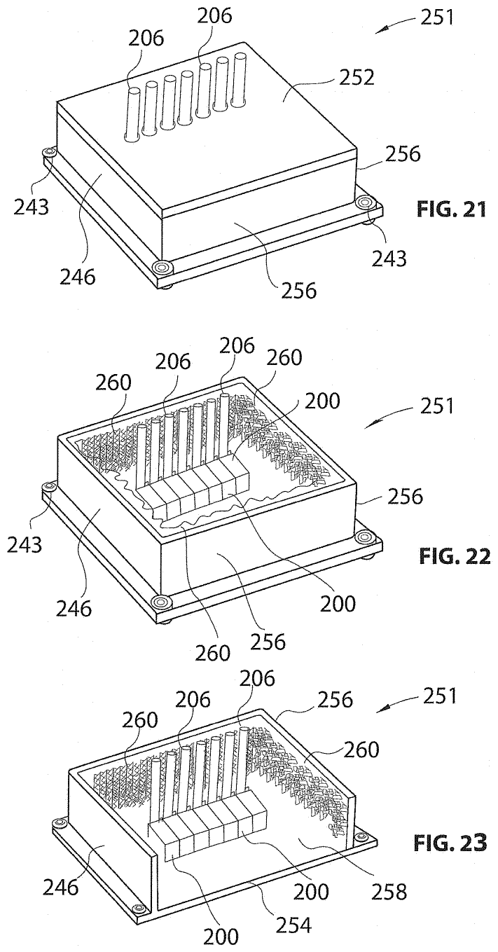

FIG. 24 illustrates a side view of the massage chair frame with oxygen generator according to the disclosure; and FIG. 25 illustrates an oxygen generator according to the disclosure.

In particular, FIG. 24 and FIG. 25 illustrate an Oxygen Concentrator Generator to provide Oxygen Therapy to the user. Generation of oxygen for a user may have a number of pleasant or beneficial effects for a user. Moreover, the user may medically need a higher percentage of oxygen in their environment for medical reasons.

FIG. 24 illustrates an exemplary positioning and arrangement of an oxygen generator 350 within the massage chair 10. In one aspect, the oxygen generator 350 may be configured as an oxygen concentrator that may concentrate oxygen from a gas supply (typically ambient air) by selectively removing nitrogen to supply an oxygen-enriched gas stream. In one aspect, the oxygen generator 350 may operate utilizing pressure swing adsorption (PSA) technology. FIG. 25 illustrates further details of the oxygen generator 350. The oxygen generator 350 may include an oxygen outlet 354, a power supply 356, mounting structures 352, a plurality of foot elements 243, and the like. The foot element 243 is preferably made by rubber to absorb and reduce the vibration noise. User can turn the oxygen generator 350 on or off via a touch button on touchscreen controller. A touch button takes input from user then sends a signal or code to the motherboard of massage chair to turn the oxygen generator on or off. The touchscreen controller and motherboard of massage chair 10 also comprise a software code so that volume of airflow is also controlled by user via a touch button on touchscreen controller. Other aspect, the armrest 316 may be designed and configured to have a control panel to control the oxygen generator on or off and oxygen volume flow. The armrest is removable for easy installation, therefor, there is a cable that connects the control panel to the motherboard having at least two sections joined together via a connector. There is an open hole on the armrest to install a connector or to run a cable through.

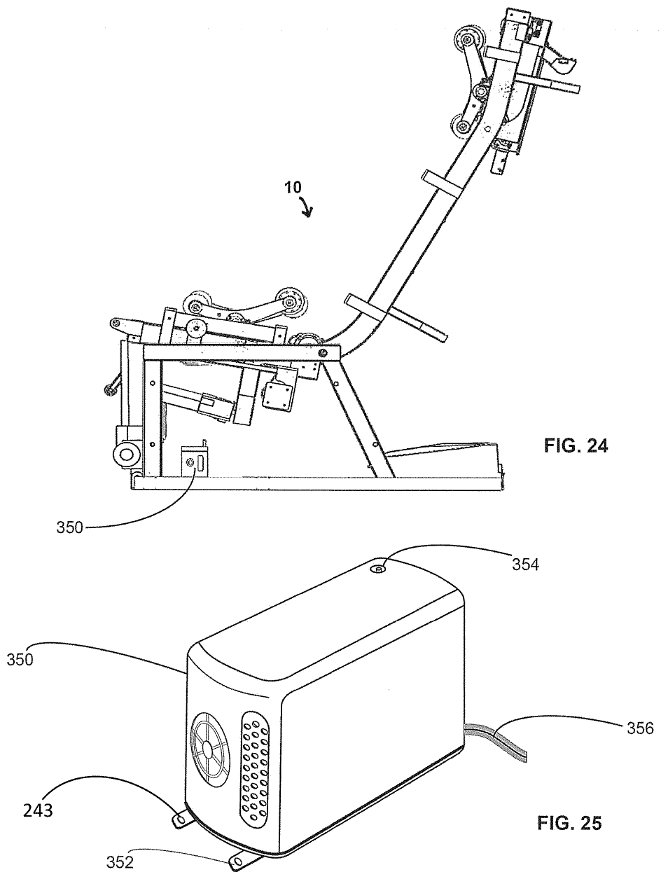

FIG. 26 illustrates a massage chair having an oxygen distribution system according to the disclosure.

In particular, FIG. 26 illustrates an oxygen distribution system that may include the oxygen generator 350 located within the massage chair 10 as illustrated in FIG. 25. Moreover, the oxygen distribution system may include an oxygen tube 362 that may be connected to the oxygen generator 350 that may extend toward the top of the massage chair 10. Additionally, the oxygen distribution system may include an oxygen tube 358 that may connect to the oxygen tube 362. In one aspect, there may be a manifold connecting the oxygen tube 358 to the oxygen tube 362. In one aspect, one or more oxygen vents 360 may be positioned at the end of the oxygen tube 358. The oxygen vents 360 may be built in the headrest or inside the massage chair 10 or located near a headrest portion of the massage chair 10. Preferably, the headrest is movable and has adjustable positions to fit different body sizes. Preferably, oxygen vents 360 is located adjacent user's noses to efficiently provide oxygen to the user. In operation, the oxygen generator 350 creates an oxygen rich supply of air that is output from the oxygen outlet 354. The oxygen outlet 354 may be connected to the oxygen tube 362 delivering the oxygen rich supply of air to the oxygen tube 358 and subsequently to the oxygen vents 360 for delivery to the environment of the user near the headrest of the massage chair 10.

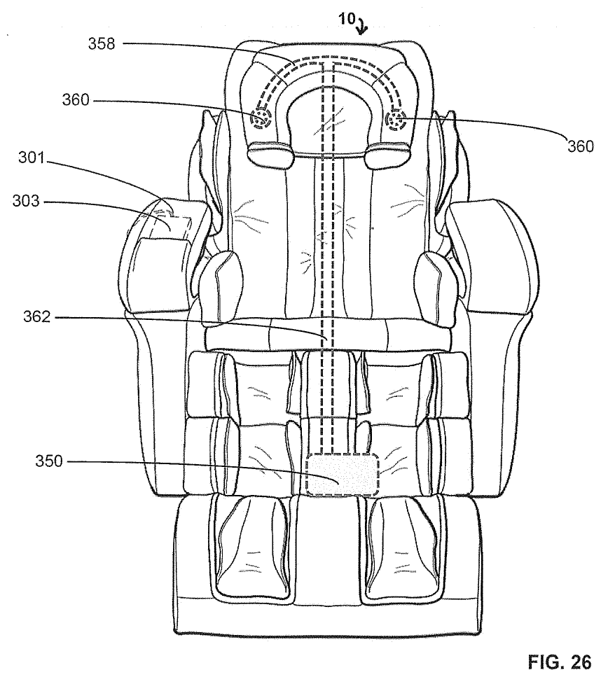

FIG. 27 illustrates a massage chair having a touchscreen remote controller, a wireless device pouch and/or a remote control pouch according to the disclosure.

In particular, FIG. 27 illustrates that the massage chair 10 may include a wireless device pouch or holder 390 and/or a remote control pouch 392. In one aspect, the remote control pouch 392 may be sized to receive a remote control for an audiovisual device such as a television, media player, DVR, cable box, stereo, and the like. The remote control pouch 392 may be formed of fabric that is the same or consistent with the massage chair 10 in order to have a pleasing and integrated appearance. In one aspect, the remote control pouch 392 may be formed of multiple connected fabric portions creating a pocket to insert the remote control into the remote control pouch 392. Other constructions are contemplated as well. In one aspect, the remote control pouch 392 may be connected to the armrest 316 in order to be easily accessed by the user of the massage chair 10.

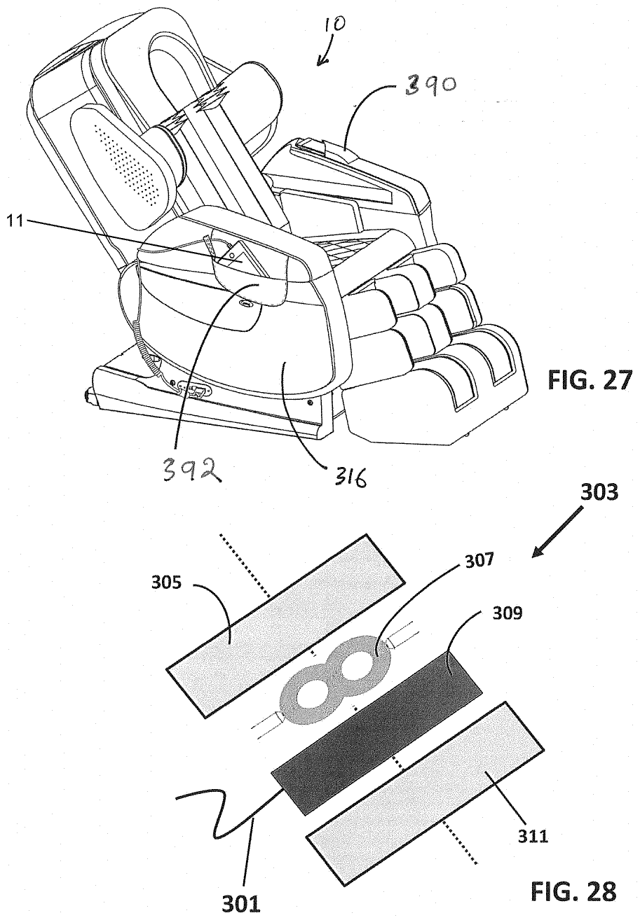

Additionally or alternatively, the massage chair 10 may include a wireless device pouch or holder 390 that may be sized to receive a wireless device such as a phone, smartphone, tablet computer, and the like. The wireless device pouch or holder 390 may be formed of fabric that is the same or consistent with the massage chair 10 in order to have a pleasing and integrated appearance. In one aspect, the wireless device pouch or holder 390 may be formed of multiple connected fabric portions creating a pocket to insert the wireless device into the wireless device pouch or holder 390. Other constructions are contemplated as well. In one aspect, the wireless device pouch or holder 390 may be connected to the armrest 316 in order to be easily accessed by the user of the massage chair 10. In a further aspect, the wireless device pouch or holder 390 may include a wireless charger 303 as described in relation to FIG. 28.

FIG. 28 illustrates a wireless charger configured to be implemented in the massage chair according to the disclosure.

In particular, the massage chair 10 may include a fast wireless charger 303 configured for charging a wireless device such as a phone, smartphone, tablet computer, and the like. In one aspect, the fast wireless charger 303 may be integrated with the wireless device pouch or holder 390. The fast wireless charger 303 may be configured as a charging pad for the wireless device. The fast wireless charger 303 may include a power connector 301 for providing power to the fast wireless charger 303 can be a pop-in and removable from the front of holder housing for easy repair and service.

In one aspect, the fast wireless charger 303 may be integrated into the massage chair 10. In one aspect, the fast wireless charger 303 may be installed to the armrest and located adjacent, under, or integrated into the wireless device pouch or holder 390. In one aspect, the fast wireless charger 303 may provide wireless power to charge a phone or similar portable devices. The fast wireless charger 303 may include a top cover 305, a coil 307, an electronic charging control printed circuit board (PCB) 309, a cable 301 to receive power, a bottom cover 311. The power for the fast wireless charger 303 may be supplied by a transformer and/or by a voltage regulator of the massage chair 10 via a power cord. Power cord is installed to the armrest and connected to a power source generated from a transformer of massage chair 10 or from a voltage regulator of motherboard of massage chair 10. For the armrest to be removable for easy installation, the power cord is preferably made of at least two sections joined together via a connector and at least an open hole on the armrest housing and a hole on the side of the massage chair 10 for power cord to run through.

In this regard, fast wireless charger 303 may transfer energy from the fast wireless charger 303 to a receiver in the back of the wireless device via electromagnetic induction. The fast wireless charger 303 may use the coil 307 implemented as an induction coil to create an alternating electromagnetic field, which a receiver coil in the wireless device converts back into electricity to be fed into the battery of the wireless device.

The fast wireless charger 303 may be configured to Intelligently Identify Charging Devices and the fast wireless charger 303 may be configured to vary the power is applied to the wireless device from 5 W to 10 W mode using QC2.0/QC3.0 adapter. In one aspect, the fast wireless charger 303 may be configured to be very light and thin, and the fast wireless charger 303 may be configured to provide good heat dissipation. Additionally, the massage chair 10 may also be configured with the fast wireless charger 303 to provide good heat dissipation.

In one aspect, the fast wireless charger 303 may be case friendly. In this regard, the fast wireless charger 303 may be configured charge straight through even extra-tough cases. Of course, such implementations may affect the charging speed a little and metal attachments or credit cards may interfere with charging.

In further aspects, the fast wireless charger 303 may be configured with High-efficiency Charging & Multiple Protection. In one aspect, the fast wireless charger 303 may be configured with a charging speed that is up to 20% faster than prior art wireless chargers. In further aspects, the fast wireless charger 303 may be configured with temperature protection, input voltage protection, input current protection, short circuit protection, and other multiple safety protection, to ensure the safety of the wireless device and the massage chair 10 during the entire charging process.

In further aspects, the fast wireless charger 303 may include a soft breathing light. In this regard, the fast wireless charger 303 may implement the soft breathing light after entering the charging mode and the soft breathing light will slowly pulse indicating charging of the wireless device by the fast wireless charger 303.

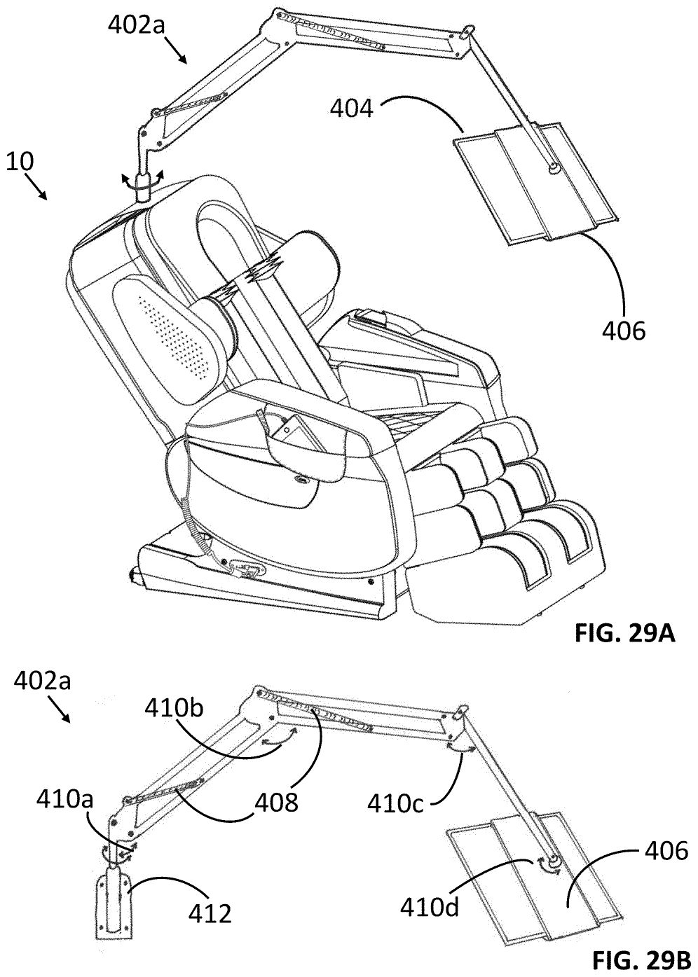

FIG. 29A illustrates a massage chair 10 having a media player holder 402a to provide free hand for user enjoying massage chair and watching favorites medias. In particular, for extending purpose, media player holder 402a may have more than one section joined together. The media player holder 402a may also have a holding mechanism 406, media player 404, and multiple springs to support and prevent media player holder 402a sagging down. Media player 404 can be a cell phone or tablet or any smart devices.

FIG. 29B illustrates a perspective view of media player holder 402a. In particular, media player holder 402a may have multiple springs 408, mounting mechanism 412, and holding mechanism 406. Mounting mechanism 412 is designed so that the media player holder 402a (or 402b) can rotate 360 degree and quickly and easily remove (or detach) from the chair when it is not in use. Mounting mechanism 412 may have single part or multiple parts assembled together. There are several screws to mount the mounting mechanism 412 to the massage chair frame. The angles 410a, 410b, and 410c can adjust from zero degree to 180 degree while the angle 410d can rotate 360 degree.

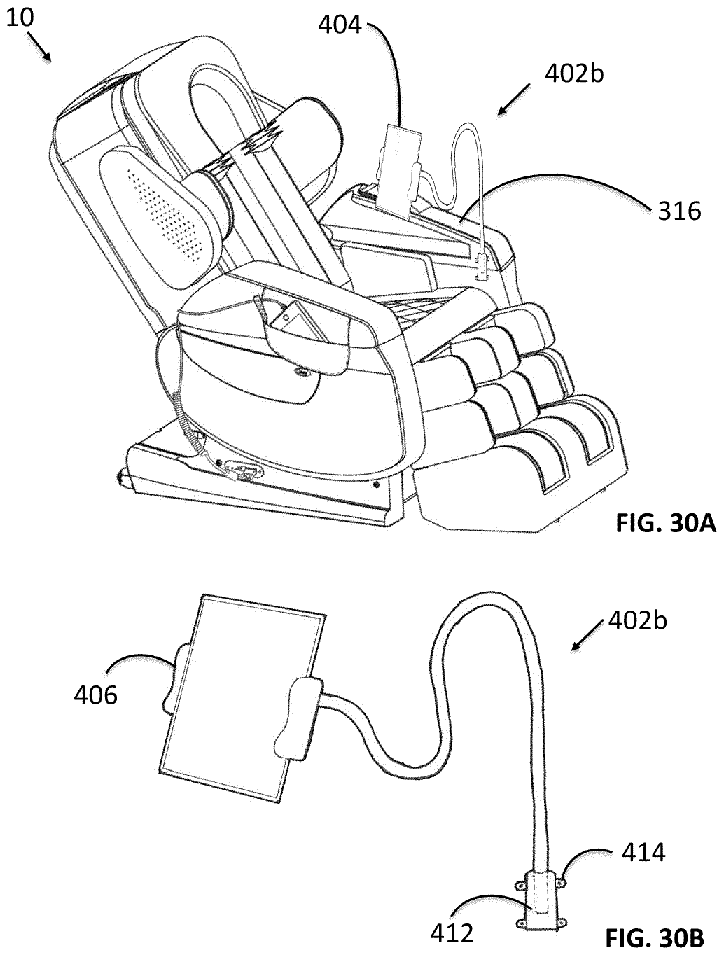

FIG. 30A illustrates a massage chair 10 having another media player holder 402b to provide free hand for user enjoying massage chair and watching favorites medias. In this regard, media player holder 402b is a flexible arm and mounted to the armrest 316 of massage chair 10 for easy assessing the chair. Media player 404 can be a cell phone or tablet or any smart devices.

FIG. 30B illustrates a perspective view of media player holder 402b. In particular, media player holder 402b may have a flexible arm, a mounting mechanism 412, and holding mechanism 406. The flexible arm can be bent to any position and any angle desired. The mounting mechanism 412 may have plurality tab and screw hole 414 to mount to the armrest.

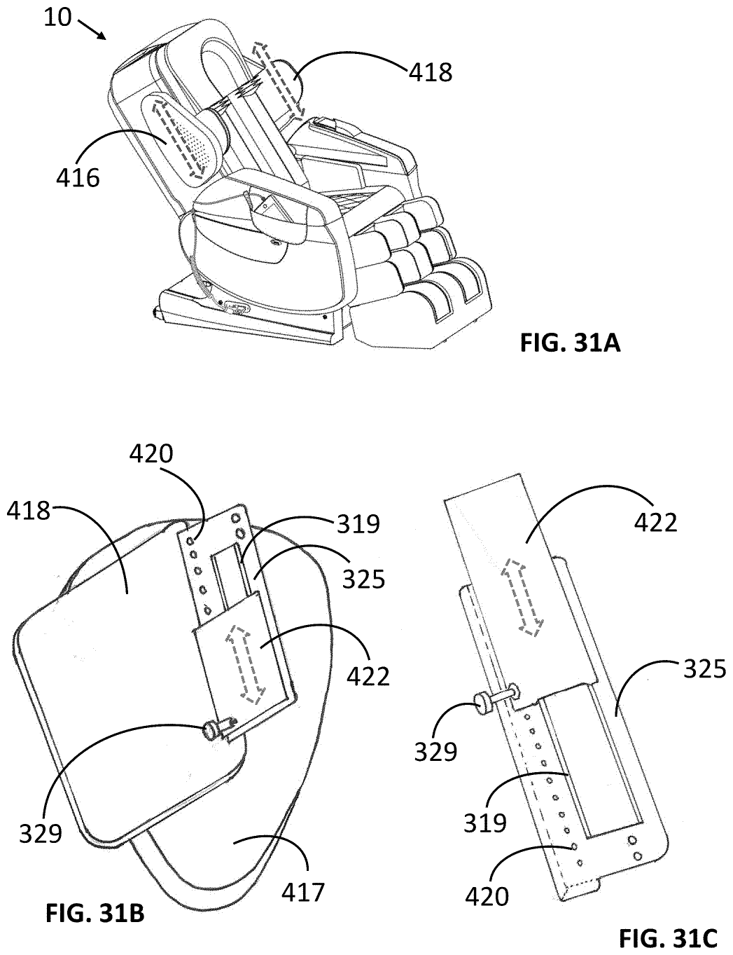

FIG. 31A illustrates massage chair 10 having shoulder supporter 417 and a slidable shoulder airbag 415 to fit user's body size and shape.

FIG. 31B illustrates a slidable shoulder airbag 415, slide rack 319, slider 422, and mounting mechanism 325. In particular, slider 422 may have a locking mechanism 329.

In further aspects, shoulder airbag 415 or shoulder supporter 417 can be mounted to the slider 422. In this case, shoulder airbag and shoulder supporter can slide together. To slide the shoulder airbag or shoulder supporter, user may pull or push the locking mechanism 329 to release the locking.

FIG. 31C illustrates slide rack 319, slider 422, and mounting mechanism 325. The slide rack 319 may have two parallel slide rails, a first slide rail 321 and a second slide rail 323. The locking mechanism 329 may have a knob, a spring, and a pointer to engage with another locking feature to lock the shoulder airbag at desired position. The locking feature can be a plurality holes 416, or spring tabs (not shown), or rubber or any high friction material to prevent the shoulder airbag moving freely. The mounting mechanism 325 may have plurality holes 416 and several holes to mount to massage chair frame.

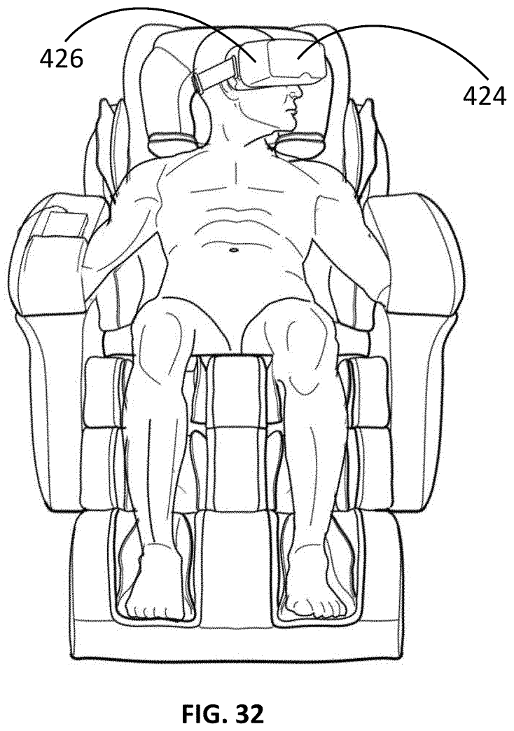

FIG. 32 illustrates a user using a virtual reality (VR) device 424 to enhance the relaxation during massage session. In particular, a holder (or housing) 426 is holding the virtual reality device 424. This holder offers the free hand to the user. User can turn the virtual reality device 424 on or off via a touch button on touchscreen controller. A touch button takes input from user then sends a signal or code to the motherboard of massage chair to turn the virtual reality device 424 on or off. Other aspect, the armrest 316 may be designed and configured to have a control panel to control the virtual reality device 424 on or off. The armrest is removable for easy installation, therefor, there is a cable that connects the control panel to the motherboard having at least two sections joined together via a connector. There is an open hole on the armrest to install a connector or to run a cable through. The virtual reality device 424 may be standalone and controlled wirelessly via a Bluetooth device. The virtual reality device 424 can be any electronic device including but not limited to a cell phone or tablet.

The disclosure has disclosed a massage chair having a massage chair frame, a massage system that includes a plurality of air massage elements, and a noise-reducing (or noise-absorbing, noise-containing or noise-cancelling) enclosure device. Additionally, the disclosure has disclosed additional features for improving user experience including armrest sliding, shoulder airbag sliding, media player holder, virtual reality player, hammering devices, heating acupuncture devices, oxygen generation, voice recognition control of the massage chair, a remote control pouch, and a wireless device pouch or holder having wireless charging functionality. All of these features provide an improved experience for the user using the massage chair 10.

The touchscreen controller 11 as described in the disclosure may include a processor, a microprocessor, a random-access memory, a read-only memory, input devices, output devices, and the like. The microprocessor may implement software for operating the various features of the massage chair 10. In one aspect a microprocessor may implement voice recognition software. The input devices may include switches, buttons, a touch sensitive screen of a touchscreen remote controller, microphones, and the like for operating the various features of the massage chair 10.

Voice recognition software may be utilized in various aspects of the systems and methods. Users may be able to vocalize, rather than utilizing other input processes. For example, the voice recognition software may be configured for generating text from voice input from a microphone or other voice input. A speech signal processor may convert speech signals into digital data that can be processed by the processor. The processor may perform several distinct functions, including serving as the speech event analyzer, the dictation event subsystem, the text event subsystem, and the executor of the application program. The speech signal processor may generate speech event data and transmit this data to the processor to be processed first by the speech event analyzer. The speech event analyzer may generate a list or set of possible candidates among the system recordings that represent or match the voice input processed by the speech signal processor. The speech event analyzer may transmit the candidate sets to a dictation event subsystem. The dictation event subsystem may analyze the candidate sets and choose the best match candidate with the highest degree of similarity. This candidate is then considered the correct translation, and the dictation event subsystem forwards the translation to the text event subsystem which in turn inputs the translated text into the device.

It is to be understood that the disclosure is not limited to the embodiments described above or as shown in the attached figures, but encompasses any and all embodiments within the spirit of the invention.

What is claimed is:

1. A massage chair comprising:
a massage chair frame;
at least one armrest;
a slide rack,
wherein said slide rack comprises a first portion, a second portion, and two parallel slide rails,
wherein said first portion is mounted to said at least one armrest while said second portion is mounted to a mounting mechanism, and
wherein at least one slide rail of said two parallel slide rails is configured to receive a bearing to reduce friction during sliding; and
said mounting mechanism configured to mount said slide rack to said massage chair frame.

2. The massage chair according to claim 1, wherein said slide rack further comprises a lock knob.

3. The massage chair according to claim 1, wherein said bearing is ball bearing.

4. The massage chair according to claim 1, wherein said mounting mechanism is configured so that said at least one armrest slides with an angle greater than zero degree compared to said massage chair frame to avoid collision of said at least one armrest with said backrest during sliding.

5. The massage chair according to claim 1, wherein said slide rack further comprises an actuator.

6. The massage chair according to claim 1, further comprising a media player holder.

7. The massage chair according to claim 1, wherein said at least one armrest is configured so that a user be able to pull said at least one armrest out then slides.

8. The massage chair according to claim 1, wherein said slide rack further comprises at least one extension section.

9. A massage chair comprising:
a backrest;
a seat;
at least one armrest;
a wireless charger,
wherein said at least one armrest comprises a wireless holder that is configured to receive said wireless charger; and
a power cord for connecting said wireless charger to a power source,
wherein said power cord comprises at least two sections joined together via a connector for easy armrest installation, and
wherein said at least one armrest comprises at least one hole for said power cord to pass through.

10. The massage chair according to claim 9, wherein said wireless charger can be accessed from a front panel for easy repair and service.

11. The massage chair according to claim 9, wherein at least one hole on a side of said at least one armrest is configured to receive a connector.

12. The massage chair according to claim 9, wherein said at least one armrest further comprises a sliding rack.

13. The massage chair according to claim 12, wherein said sliding rack comprises an actuator.

14. The massage chair according to claim 9, further comprising a media player holder.

15. A massage chair comprising:
at least one removeable armrest;
an oxygen generator or concentrator;
a removable headrest; and
a control cable,
wherein said massage chair comprises a button to activate said oxygen generator or concentrator,
wherein a housing of said at least one removeable armrest comprises at least one hole on a side for said control cable to pass through for easy armrest installation, and
wherein said removeable headrest comprises at least one vent capable of being positioned adjacent to noses of a user to efficiently distribute oxygen to the user.

16. The massage chair according to claim 15, wherein said button is push on-push off button and located on said at least one removeable armrest.

17. The massage chair according to claim 15, wherein said button is a touch button of a touchscreen controller.

18. The massage chair according to claim 15, further comprising a media player holder.

19. The massage chair according to claim 15, wherein said at least one removeable armrest further comprises a slide rack.

20. A massage chair comprising:
a backrest
at least one armrest; and
a shoulder airbag,
wherein said shoulder airbag comprises a slide rack to slide said shoulder airbag to fit body size of a user,
wherein said slide rack comprises a first portion, a second portion, and two parallel slide rails,
wherein said first portion is mounted to a sliding mounting while said second portion is mounted to a mounting mechanism,
wherein said mounting mechanism is mounted to said backrest, and
wherein at least one slide rail of said two parallel slide rails is configured for receiving a bearing to reduce friction during sliding.

21. The massage chair according to claim 20, wherein said bearing is ball bearing.

22. The massage chair according to claim 20, wherein said mounting mechanism comprises a lock feature to prevent said sliding mounting moving freely.

23. The massage chair according to claim 22, wherein said lock feature comprises a tab.

24. The massage chair according to claim 20, wherein said slide rack further comprises a lock mechanism.

25. The massage chair according to claim 24, wherein said lock mechanism further comprises a lock knob and a plurality of holes.

26. The massage chair according to claim 20, wherein said slide rack further comprises an actuator.

27. The massage chair according to claim 20, further comprising a media player holder.

28. The massage chair according to claim 27, wherein said media player holder is mounted to said at least one armrest.

29. The massage chair according to claim 27, wherein said media player holder is mounted to said backrest.

30. The massage chair according to claim 20, further comprising a hammer punching device.
